(19) United States
(12) Patent Application Publication
(10) Pub. No.: US 2025/0143868 A1
(43) Pub. Date: May 8, 2025

(54) MEDICAL DEVICE SYSTEMS AND METHODS INCLUDING IMPLANT DELIVERY DEVICE

(71) Applicants: Smith & Nephew, Inc., Memphis, TN (US); Smith & Nephew Orthopaedics AG, Zug (CH); Smith & Nephew Asia Pacific Pte. Limited

(72) Inventors: Cori G. Pierce, Salem, NH (US); Brendan Jablonski, Boston, MA (US)

(73) Assignees: Smith & Nephew, Inc., Memphis, TN (US); Smith & Nephew Orthopaedics AG, Zug (CH); Smith & Nephew Asia Pacific Pte. Limited, (omitted)

(21) Appl. No.: 18/934,810

(22) Filed: Nov. 1, 2024

Related U.S. Application Data
(60) Provisional application No. 63/595,799, filed on Nov. 3, 2023.
(60) Provisional application No. 63/550,212, filed on Feb. 6, 2024.

(51) Int. Cl. A61F 2/08

(52) U.S. Cl. CPC A61F 2/0805

(57) ABSTRACT

An implant delivery device may include a handle, an elongate shaft extending distally from the handle, and a support frame coupled to a distal end of the shaft. The shaft includes a lumen extending along an entire length of the shaft. The lumen opens to a proximal end of the handle and is configured to receive a bone anchor insertion device having a bone anchor thereon. A medical device system includes the implant delivery device and a bone anchor insertion device advanceable through the lumen of the shaft to secure an implant coupled to the support frame to a bone. A method may include advancing the implant to a treatment site with the implant delivery device, deploying the implant with the support frame, and advancing the bone anchor through the shaft to secure the implant to bone while the support frame remains coupled to the shaft and the implant.

### CROSS REFERENCE TO RELATED APPLICATIONS

This application claims the benefit of U.S. Provisional Patent Application Ser. No. 63/595,799 filed on Nov. 3, 2023, and U.S. Provisional Patent Application Ser. No. 63/550,212 filed on Feb. 6, 2024, the disclosures of which are incorporated herein by reference.

### TECHNICAL FIELD

The present disclosure pertains generally to medical devices and methods of using medical devices. More particularly, the present disclosure relates to medical devices and/or systems, and methods of using the same, for securing a medical implant to tissue in an operative space in a body of a patient

### BACKGROUND

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Current procedures for treatment of a torn tendon include affixing a biocompatible implant over the torn tendon. There is an ongoing need to deliver and adequately secure medical implants during an arthroscopic procedure in order to treat injuries to the rotator cuff, rotator cuff tendons, or other soft tissue or tendon injuries throughout a body.

### SUMMARY

This disclosure provides design, material, manufacturing method, and use alternatives for medical devices, including systems for delivering a medical implant along with bone and/or tissue anchors into an operative space.

A first embodiment is an implant delivery device. The Implant delivery device includes a handle, an elongate shaft extending distally from the handle, and a support frame coupled to a distal end of the elongate shaft. The elongate shaft includes a lumen extending along an entire length of the elongate shaft. The lumen opens to a proximal end of the handle. The lumen of the elongate shaft is configured to receive a bone anchor insertion device having a bone anchor thereon through the lumen of the elongate shaft.

Alternatively or additionally to any of the examples herein, in another example, the implant delivery device includes an outer sleeve that is disposed about the elongate shaft and is axially slidable relative to the elongate shaft.

Alternatively or additionally to any of the examples herein, in another example, the implant delivery device includes an implant sheath fixedly attached to a distal end of the outer sleeve.

Alternatively or additionally to any of the examples herein, in another example, the support frame is releasably coupled to an implant and the implant sheath is configured to constrain the support frame and the implant in a delivery configuration.

Alternatively or additionally to any of the examples herein, in another example, the support frame and the implant are configured to expand for deployment at a treatment site when unconstrained by the implant sheath.

Alternatively or additionally to any of the examples herein, in another example, the support frame is configured to permit movement of the distal end of the elongate shaft relative to the implant after the implant is secured to bone at the treatment site while coupled to the implant.

Alternatively or additionally to any of the examples herein, in another example, the support frame is configured to permit the distal end of the elongate shaft to move over an entirety of an upper surface of the implant after the implant is secured to bone at the treatment site while coupled to the implant.

Alternatively or additionally to any of the examples herein, in another example, the handle includes an actuator configured to slide the outer sleeve axially relative to the elongate shaft.

Alternatively or additionally to any of the examples herein, in another example, the handle includes a retractor configured to remove a distal end of the bone anchor insertion device from bone.

Another example is a medical device system. The system includes an implant delivery device and a bone anchor insertion device. The implant delivery device includes a handle, an elongate shaft extending distally from the handle, and a support frame coupled to a distal end of the elongate shaft. The elongate shaft includes a lumen extending along an entire length of the elongate shaft. The lumen opens to a proximal end of the handle. T bone anchor insertion device has a bone anchor mounted on a distal end thereof. The bone anchor and the bone anchor insertion device are advanceable through the lumen of the elongate shaft to secure an implant coupled to the support frame to a bone with the bone anchor.

Alternatively or additionally to any of the examples herein, in another example, the system includes a tissue anchor delivery device including at least one tissue anchor. The tissue anchor delivery device is advanceable through the lumen of the elongate shaft to secure the implant coupled to the support frame to tissue adjacent the bone with the at least one tissue anchor.

Alternatively or additionally to any of the examples herein, in another example, the support frame is configured to permit the distal end of the elongate shaft to move relative to the implant after the implant is secured to the bone with the bone anchor.

Alternatively or additionally to any of the examples herein, in another example, the support frame extends distally from the distal end of the elongate shaft.

Alternatively or additionally to any of the examples herein, in another example, the bone anchor insertion device is positionable at an oblique angle relative to an upper surface of the implant to secure the implant coupled to the support frame to the bone with the bone anchor.

Another example is a method of attaching an implant at a treatment site. The method includes advancing the implant to the treatment site with an implant delivery device and then deploying the implant at the treatment site with a support frame coupled to a distal end of an elongate shaft of the implant delivery device. A bone anchor is then advanced on a distal end of a bone anchor insertion device through the elongate shaft of the implant delivery device to secure the implant to bone with the bone anchor while the support frame remains coupled to the elongate shaft and the implant.

Alternatively or additionally to any of the examples herein, in another example, deploying the implant at the treatment site comprises sliding an outer sleeve of the implant delivery device proximally relative to the elongate shaft and the support frame using an actuator of the implant delivery device.

Alternatively or additionally to any of the examples herein, in another example, the method includes removing the distal end of the bone anchor insertion device from bone with a retractor disposed in a handle of the implant delivery device, and then removing the bone anchor insertion device from the elongate shaft of the implant delivery device.

Alternatively or additionally to any of the examples herein, in another example, the method includes inserting a tissue anchor delivery device including at least one tissue anchor through the elongate shaft to secure the implant to tissue adjacent the bone with the at least one tissue anchor while the support frame remains coupled to the elongate shaft and the implant after removing the bone anchor insertion device from the elongate shaft of the implant delivery device.

Alternatively or additionally to any of the examples herein, in another example, the implant is secured to tissue adjacent the bone with the at least one tissue anchor at an opposite end of the implant from the bone anchor while the support frame remains coupled to the elongate shaft and the implant.

Alternatively or additionally to any of the examples herein, in another example, the distal end of the elongate shaft is configured to pierce the implant and engage the bone to temporarily fix the implant to the bone as the bone anchor secures the implant to the bone while the support frame remains coupled to the elongate shaft and the implant.

The above summary of some embodiments, aspects, and/or examples is not intended to describe each disclosed embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description in connection with the accompanying drawings, in which:

FIGS. 1-4 schematically illustrate selected aspects of a prior art system for delivering and securing an implant at a treatment site;

FIGS. 5-10 schematically illustrate selected aspects of a medical device system and/or an implant delivery device according to the disclosure;

FIGS. 11-14 are cross-sectional views illustrating selected aspects of alternative configurations of the implant delivery device;

FIGS. 15-16 schematically illustrate selected aspects of an alternative configuration of the medical device system and/or the implant delivery device shown in FIGS. 8-9; and

FIG. 17 schematically illustrates selected aspects of an alternative configuration of the implant delivery device.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. Like reference numerals indicate like elements throughout the views. The detailed description and drawings are intended to illustrate but not limit the disclosure. Those skilled in the art will recognize that the various elements described and/or shown may be arranged in various combinations and configurations without departing from the scope of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, enlightened by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

It is noted that references in the specification to “an embodiment”, “some embodiments”, “other embodiments”, etc., indicate that the embodiment(s) described may include one or more particular features, structures, and/or characteristics. However, such recitations do not necessarily mean that all embodiments include the particular features, structures, and/or characteristics. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, individual elements described herein, even if not explicitly shown in a particular combination, are contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The figures generally illustrate selected components and/or arrangements of medical devices, systems, and/or methods. It should be noted that in any given figure, some features may not be shown, or may be shown schematically, for simplicity. Additional details regarding some elements may be illustrated in other figures in greater detail. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to features or elements may be equally referred to all instances and quantities beyond one of said feature or element. As such, it will be understood that the following discussion may apply equally to any and/or all of the elements for which there are more than one within the medical devices, systems, and/or methods, unless explicitly stated to the contrary. Additionally, all instances of some elements or features may not be shown in each figure for clarity.

With its complexity, range of motion, and extensive use, a common soft tissue injury is damage to the rotator cuff or rotator cuff tendons. Damage to the rotator cuff is a potentially serious medical condition that may occur during hyperextension, from an acute traumatic tear, or from overuse of the joint. An accepted treatment for rotator cuff tears may include reattaching the torn tendon to the humeral head using sutures. Additionally, in treating rotator cuff tears, an accepted practice may also include the placement of a scaffold over the repaired tendon to mechanically reinforce the repaired tendon and/or to promote tissue reformation. Therefore, there is an ongoing need to deliver, position, and secure medical implants to soft tissue during an arthroscopic procedure in order to treat injuries to the rotator cuff, rotator cuff tendons, or other soft tissue or tendon injuries throughout a body. At least some of those procedures may involve the placement of multiple surgical anchors into and/or associated with a medical implant, and in some cases may involve multiple devices. There is an ongoing need to deliver a medical implant along with multiple surgical anchors within a single procedure while minimizing device removal from and insertion into the patient.

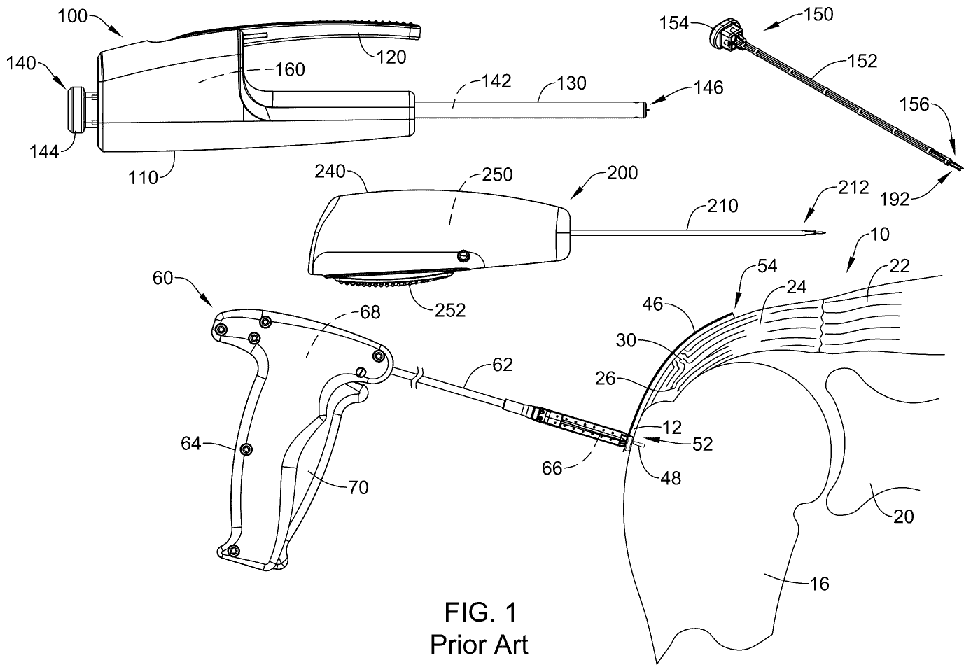

For reference, selected aspects of a shoulder 10 including an implant 12 positioned at a treatment site within an operative space in a body of a patient are illustrated and/or identified in FIG. 1. Some aspects of the shoulder 10, such as the patient's skin, are not shown, and/or some aspects of the shoulder 10 are not shown or explicitly called out in subsequent figures to improve clarity. In at least some embodiments, the implant 12 may be a sheet-like implant and/or a scaffold configured to reinforce damaged tissue. In some embodiments, the implant 12 may include collagen and/or may be collagen based. The shoulder 10 includes a head of the humerus 16 mating with a glenoid fossa of the scapula 20. The glenoid fossa includes a shallow depression in the scapula 20. A supraspinatus tendon 22 is shown and a distal tendon 24 of the supraspinatus tendon 22 meets the humerus 16 at an insertion point 26. As illustrated, the distal tendon 24 includes a damaged portion located near the insertion point 26. The damaged portion includes a tear 30 extending partially through the distal tendon 24. In some cases, the tear 30 may be a partial thickness tear, as illustrated in FIG. 1. In some cases, the tear 30 may be a complete thickness tear. The tear 30 in FIG. 1 is depicted on the bursal side of the distal tendon 24, however, the tear 30 may also be on the opposite or articular side of the distal tendon 24 and/or may include internal tears to the distal tendon 24 not visible on or from either side.

In FIGS. 1-4, several aspects of a prior art system for delivering the implant 12 along with bone and/or tissue anchors are illustrated. FIGS. 1-3 illustrate that the implant 12 has been placed over the tear 30. In this example, the implant 12 is placed on the bursal side of the distal tendon 24 regardless of whether the tear is on the bursal side, the articular side, or within the distal tendon 24. Further, the implant 12 may overlay multiple tears.

Delivery of the implant 12 to a treatment site of a patient typically requires a physician to create a plurality of incisions in the patient (e.g., in the patient's skin, etc.) sufficient to access the treatment site and/or the operative space. The prior art system includes an implant delivery device 60 including an outer shaft 62 configured to deliver the implant 12 into the operative space. After creating the access site, the physician inserts the implant delivery device 60 through a first incision into the operative space (e.g., subcutaneously and/or under the skin) and positions a distal end of the implant delivery device 60 adjacent the treatment site and/or adjacent tissue (e.g., tendon, muscle, etc.) within the operative space. In the illustrated example, the implant 12 is advanced into the operative space within the outer shaft 62 of the implant delivery device 60.

The implant delivery device 60 includes a proximal handle 64 coupled to a proximal end of the outer shaft 62. A support frame 46 is coupled to an inner shaft 66 of the implant delivery device 60. In some embodiments, the support frame 46 may be detachably coupled to the inner shaft 66. The support frame 46 is detachably coupled to the implant 12 via attachment arms 47. The support frame 46 and the implant 12 are contained within the outer shaft 62 of the implant delivery device 60 during delivery. A portion of the support frame 46 and a portion of the implant 12 are wrapped around the inner shaft 66 during delivery. The proximal handle 64 includes an actuation mechanism 68 configured to move the outer shaft 62 proximally relative to the inner shaft 66 and the proximal handle 64 to deploy and release the implant 12 and the support frame 46. The implant delivery device 60 includes a trigger 70 operatively coupled to the actuation mechanism 68 such that actuation of the trigger 70 operates the actuation mechanism 68 to move the outer shaft 62 proximally relative to the inner shaft 66 and the proximal handle 64. The implant delivery device 60 is manipulated to position and deploy the implant 12 at the treatment site.

When positioning and deploying the implant 12 at the treatment site, the physician orients the implant 12 such that a proximal portion 52 of the implant 12 is overlaid on a portion of the humerus 16 (e.g., on the bone), while a distal portion 54 of the implant 12 is overlaid on the distal tendon 24. In some embodiments, the support frame 46 may include a tack member 48 extending therefrom and extending through the implant 12. The tack member 48 is configured to provide temporary fixation of the support frame 46 and the implant 12 to bone or tissue at the treatment site.

In embodiments using the tack member 48, the outer shaft 62 and the inner shaft 66 are removable from the operative space after deploying the support frame 46 and the implant 12, but removal does not necessarily occur in each instance. The support frame 46 and the implant 12 may be detached from the inner shaft 66 prior to removing the outer shaft 62 and the inner shaft 66 from the operative space, thereby leaving the implant 12 and the support frame 46 disposed at the treatment site.

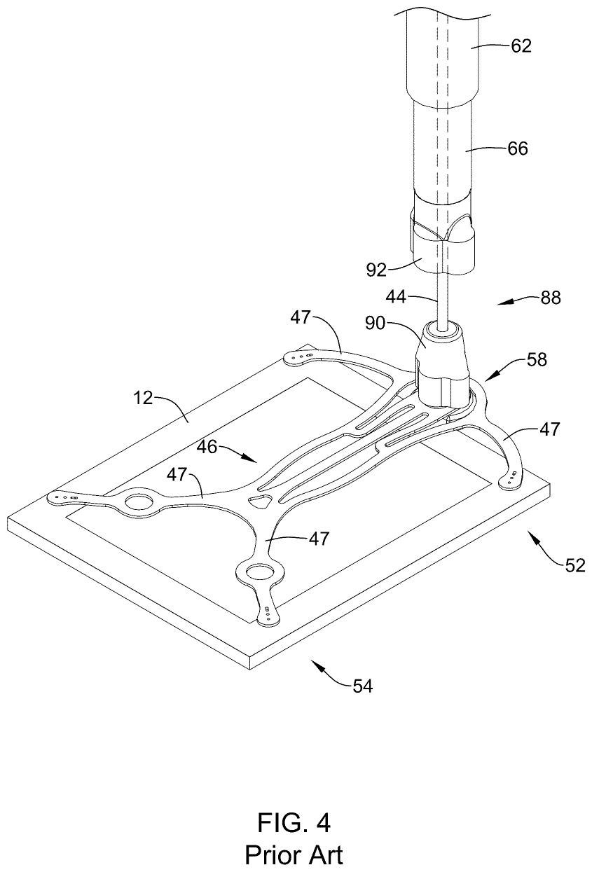

A tether 44 (e.g., FIG. 4) extends proximally from the support frame 46. The tether 44 is coupled to the support frame 46 at a head portion 58 of the support frame 46 via a connection assembly 88. The connection assembly 88 includes a first connection member 90 attached to the head portion 58 of the support frame 46 and a second connection member 92 attached to a distal end 50 of the inner shaft 66. The tether 44 extends through the inner shaft 66 to a free end that is disposed outside of the operative space after the implant delivery device 60 has been removed from the operative space. The tether 44 is pulled proximally to remove the support frame 46 and/or the tack member 48 from the implant 12 and/or the operative space after the implant 12 has been affixed to bone and/or tissue at the treatment site.

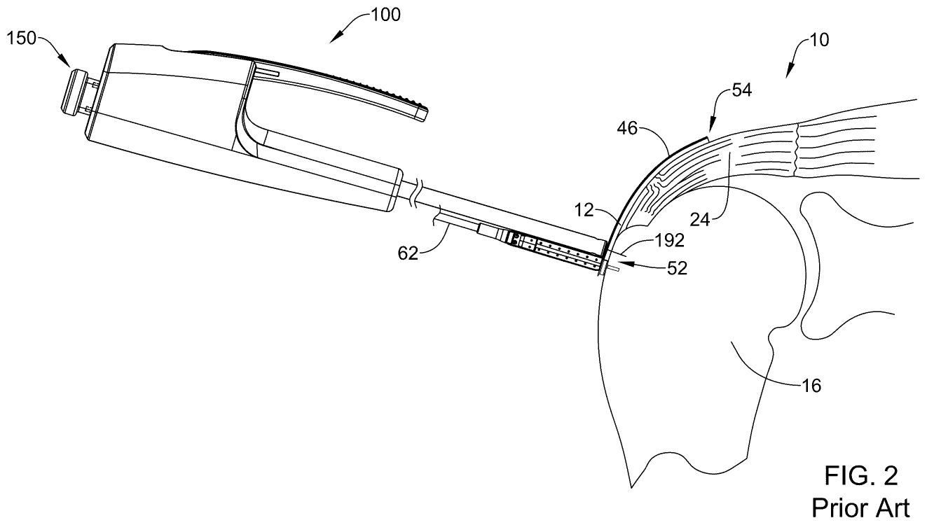

The prior art system includes a bone anchor delivery device 100. The bone anchor delivery device 100 is advanced and/or inserted through the incision(s) into the operative space, as seen in FIG. 2 for example. Once the implant 12 has been placed appropriately, the bone anchor delivery device 100 inserts a bone anchor (or multiple bone anchors) through the proximal portion 52 of the implant 12 into bone (e.g., the head of the humerus 16) at the treatment site and/or within the operative space. In embodiments, where the tack member 48 is used, the tack member 48 is generally centered within the proximal portion 52 and the bone anchors 192 are inserted on opposite sides of the tack member 48 to secure the proximal portion 52 of the implant 12 to bone (e.g., the head of the humerus 16) at the treatment site.

The bone anchor delivery device 100 includes a handle assembly 110, a lever 120 movably coupled to the handle assembly 110, and a sheath 130 extending distally from the handle assembly 110. The bone anchor delivery device 100 includes a bone punch 140 and a bone anchor insert 150, as seen in FIG. 1. The bone punch 140 is configured to form one or more holes in the bone (e.g., the head of the humerus 16). The bone anchor insert 150 is configured to insert the bone anchor(s) 192 into the one or more holes formed in the bone with the bone punch 140. The bone punch 140 includes an elongate shaft 142, a head 144 disposed at a proximal end of the elongate shaft 142, and a piercing tip 146 disposed at a distal end of the elongate shaft 142. The bone punch 140 and/or the piercing tip 146 may include two piercing tips. The piercing tip 146 is configured to pierce, to penetrate, and/or to be driven into bone (e.g., the head of the humerus 16). For example, a distal force may be applied to the head 144 of the bone punch 140 with a mallet to urge the piercing tip 146 into bone (e.g., the head of the humerus 16). The bone anchor insert 150 includes an elongate shaft 152, a head 154 disposed at a proximal end of the elongate shaft 152, and one or more arms 156 disposed at a distal end of the elongate shaft 152 to retain a bone anchor 192 (e.g., FIG. 2) for insertion into the bone (e.g., the head of the humerus 16).

The bone anchor delivery device 100 includes a retraction mechanism 160 disposed within the handle assembly 110 and engaged with the lever 120. After the piercing tip 146 of the bone punch 140 is driven into bone (e.g., the head of the humerus 16), the lever 120 is moved relative to the handle assembly 110 to actuate the retraction mechanism 160 to remove the piercing tip 146 from the bone (e.g., the head of the humerus 16). Thereafter, the bone punch 140 is removed from the handle assembly 110, while holding the handle assembly 110 and the sheath 130 in place, and the bone anchor insert 150 is inserted through the handle assembly 110 and the sheath 130 in its place to insert the bone anchor(s) 192 into the one or more holes formed in the bone (e.g., the head of the humerus 16) by the piercing tip 146 of the bone punch 140, as seen in FIG. 2. The bone anchor insert 150 is then removed from the bone anchor delivery device 100. If desired, the lever 120 and the retraction mechanism 160 may be used to remove the bone anchor insert 150. The process may be repeated in different locations within the operative space to deploy as many bone anchors as desired.

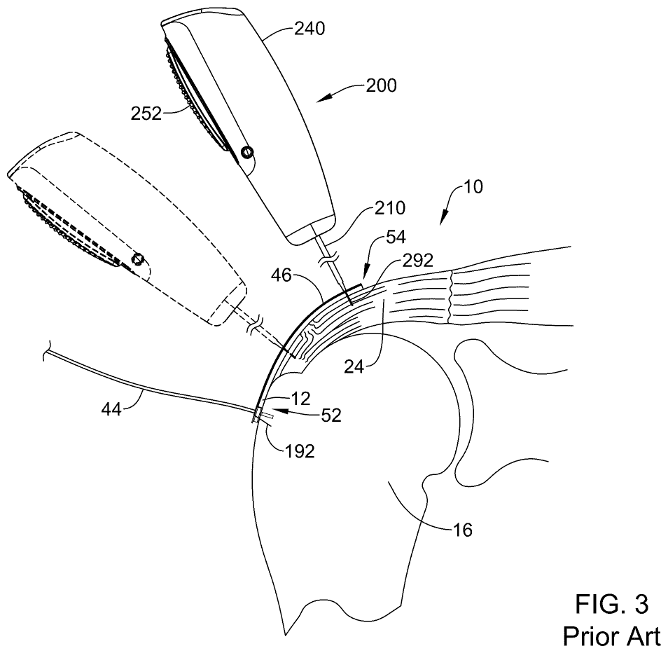

The prior art system includes a tissue anchor delivery device 200. The tissue anchor delivery device 200 is advanced and/or inserted through the incisions into the operative space, as seen in FIG. 3 for example. Once the implant 12 has been placed appropriately, the tissue anchor delivery device 200 inserts a tissue anchor (or multiple tissue anchors) through the implant 12 into tissue (e.g., the distal tendon 24) at the treatment site and/or within the operative space. The tissue anchor(s) 292 (e.g., FIG. 3) are disposed around a periphery of the implant 12 to secure the implant 12 to tissue (e.g., the distal tendon 24) at the treatment site.

The tissue anchor delivery device 200 includes an elongate shaft 210 having a lumen extending therein to a distal end 212 of the elongate shaft 210. The tissue anchor delivery device 200 includes a handle 240 disposed at and/or coupled to a proximal end of the elongate shaft 210. The handle 240 includes an actuation mechanism 250 configured to advance the tissue anchor(s) 292 out of the elongate shaft 210 and/or into the implant 12 and/or tissue at the treatment site and/or within the operative space. The actuation mechanism 250 is disposed within an interior of the tissue anchor delivery device 200, the handle 240, and/or the elongate shaft 210.

The elongate shaft 210 includes two tines extending distally away from the distal end 212. The two tines extend parallel to the central longitudinal axis of the elongate shaft 210. Collectively, the two tines define and/or border a passage through which the tissue anchor(s) 292 may pass through as the tissue anchor(s) 292 is advanced and/or deployed out of the elongate shaft 210. The two tines create pilot holes at the treatment site (e.g., within tissue and/or the implant 12) via application of a distal force to the tissue anchor delivery device 200, wherein the tissue anchor(s) 292 are inserted into the pilot holes upon actuation of the actuation mechanism 250.

The tissue anchor delivery device 200 and/or the actuation mechanism 250 includes a trigger 252. The trigger 252 is movable relative to the handle 240 to actuate the actuation mechanism 250. The trigger 252 may be a lever, and is illustrated in FIGS. 1 and 3 in an actuated position (e.g., after actuation of the actuation mechanism 250), wherein the trigger 252 has been squeezed and/or moved toward and/or into the handle 240.

After the desired bone anchors 192 and tissue anchors 292 have been deployed, the tether 44 is pulled to remove the support frame 46 and tack member 48 (where present) from the operative space, thereby leaving the implant 12 secured to bone and tissue within at the treatment site and/or within the operative space.

As may be seen from FIGS. 1-4, the prior art system for implanting the implant 12 is extensive and time consuming. Several incisions are required, each with its own inherent risks and/or consequences (e.g., patient discomfort, incision closure, infection, etc.). This disclosure offers an alternative implant delivery device and/or an alternative medical device system that may reduce the number of different tools and/or instruments required, and/or may reduce the number of incisions needed to perform the procedure, thereby speeding up the procedure and/or reducing risk and/or discomfort to the patient.

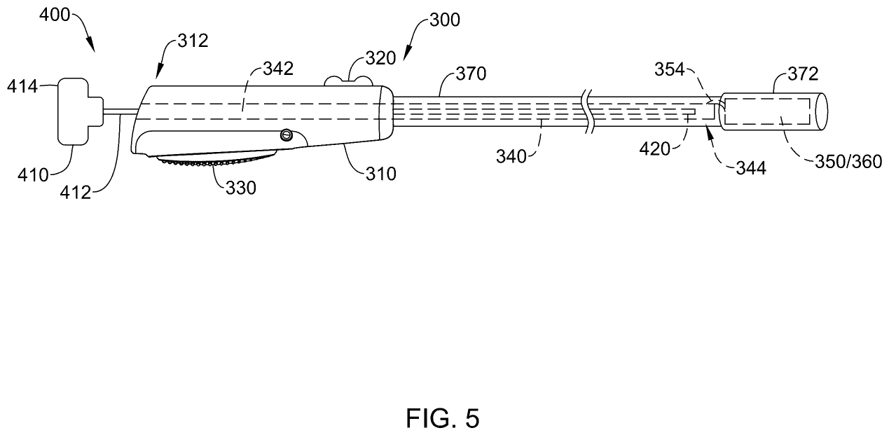

FIGS. 5-10 schematically illustrate selected aspects of an implant delivery device 300 and/or a medical device system 400 according to the disclosure. In some embodiments, the implant delivery device 300 may comprise a handle 310. In at least some embodiments, the handle 310 may be shaped, sized, and configured to be gripped, manipulated, and/or operated by a single hand of a user. Other configurations are also contemplated.

In some embodiments, the handle 310 may comprise an actuator 320. In some embodiments, the actuator 320 may at least partially extend from the handle 310. In some embodiments, the actuator 320 may comprise a slider knob configured to move and/or slide relative to the handle 310. In some embodiments, the actuator 320 may comprise a trigger configured to move relative to the handle 310. In some embodiments, the actuator 320 may comprise a movable button configured to move relative to the handle 310. In some embodiments, the actuator 320 may comprise a touch sensitive button or sensor disposed on the handle 310. Other configurations are also contemplated.

Additional details related to the actuator 320 are described herein.

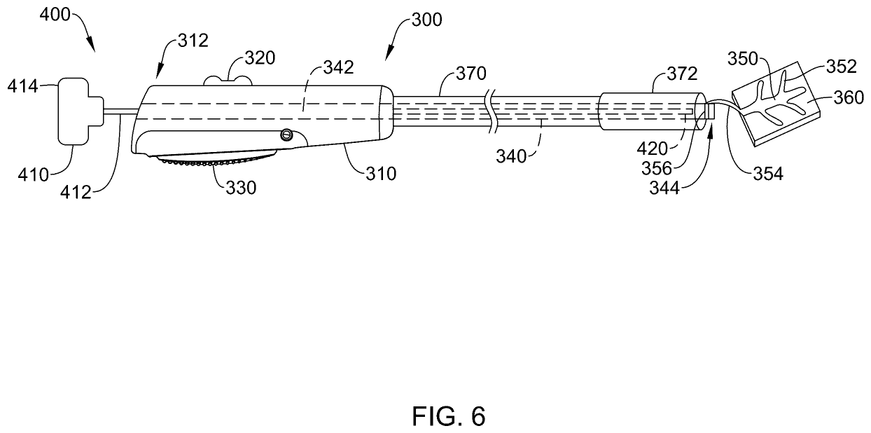

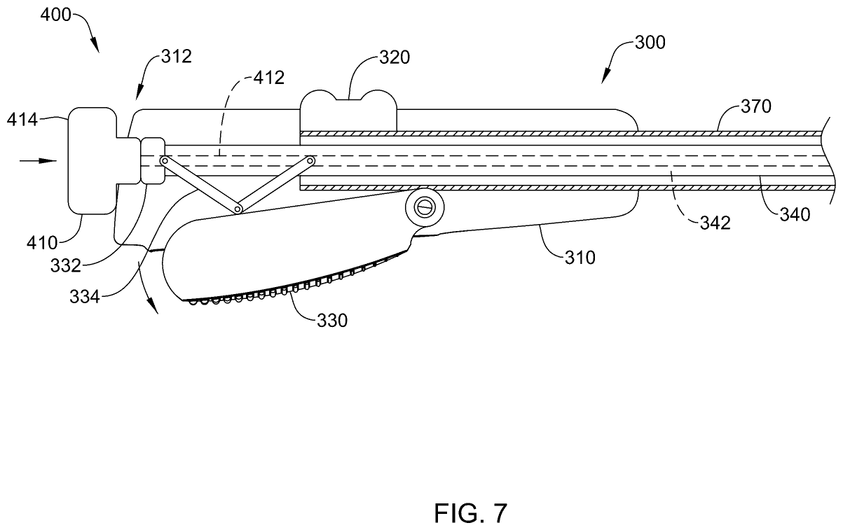

In some embodiments, the handle 310 may comprise a retractor 330 (e.g., FIG. 7). In some embodiments, the retractor 330 may at least partially extend from the handle 310. In some embodiments, the retractor 330 may be configured to move between an extended position and an ejection position. In some embodiments, the retractor 330 may comprise a lever configured to move and/or pivot relative to the handle 310 between the extended position and the ejection position. For reference, in FIGS. 5-6, the retractor 330 is shown in the ejection position, and in FIG. 7, the retractor 330 is shown in the extended position. In some embodiments, the retractor 330 may comprise a trigger configured to move relative to the handle 310. In some embodiments, the retractor 330 may comprise a movable button configured to move relative to the handle 310. In some embodiments, the retractor 330 may comprise a touch sensitive button or sensor disposed on the handle 310. Other configurations are also contemplated. Additional details related to the retractor 330 are described herein.

In some embodiments, the implant delivery device 300 may comprise an elongate shaft 340 extending distally from the handle 310. In some embodiments, the elongate shaft 340 may include a lumen 342 extending along and/or within the elongate shaft 340. In some embodiments, the lumen 342 may extend along and/or within an entire length of the elongate shaft 340. In at least some embodiments, the lumen 342 may open to a proximal end 312 of the handle 310. In some embodiments, the lumen 342 may open to an exterior of the handle 310 at and/or adjacent the proximal end 312 of the handle 310. In some embodiments, the elongate shaft 340 may extend to the proximal end 312 of the handle 310. In some embodiments, the elongate shaft 340 may be secured relative to the handle 310. In some embodiments, the elongate shaft 340 may be fixedly attached to the handle 310. Other configurations are also contemplated.

In some embodiments, the implant delivery device 300 may comprise a support frame 350 coupled to a distal end 344 of the elongate shaft 340, as seen in FIG. 6. In at least some embodiments, the support frame 350 may extend distally from the distal end 344 of the elongate shaft 340. In some embodiments, the support frame 350 may be releasably couplable to an implant 360. In some embodiments, the support frame 350 may comprise a plurality of attachment arms 352 configured to releasably couple to the implant 360. In some embodiments, the implant 360 may correspond to and/or may be the implant 12 discussed above and/or the plurality of attachment arms 352 may correspond to and/or may be the attachment arms 47 discussed above. In some embodiments, the implant 360 may be a sheet-like implant and/or a scaffold configured to reinforce damaged tissue. In some embodiments, the implant 360 may include collagen and/or may be collagen based. Other configurations are also contemplated. In at least some embodiments, the implant delivery device 300 may comprise the implant 360 and/or the support frame 350 may be releasably coupled to the implant 360. The support frame 350 and the implant 360 may extend distally from and/or may be disposed distally of the distal end 344 of the elongate shaft 340.

In some embodiments, the support frame 350 may be fixedly attached to the distal end 344 of the elongate shaft 340. In some embodiments, the support frame 350 may comprise a proximal extension 354 coupled to the distal end of the elongate shaft 340. In some embodiments, the proximal extension 354 of the support frame 350 may be fixedly attached to the distal end 344 of the elongate shaft 340. In some embodiments, the support frame 350 and/or the proximal extension 354 may be fixedly attached to the distal end 344 of the elongate shaft 340 at a collar 356. In some embodiments, the collar 356 may extend circumferentially around a majority of the distal end 344 of the elongate shaft 340. In some embodiments, the collar 356 may extend completely around a majority of the distal end 344 of the elongate shaft 340. In some embodiments, the collar 356 may be at least partially embedded within the distal end 344 of the elongate shaft 340. In some embodiments, the collar 356 may be fixedly attached to the distal end 344 of the elongate shaft 340 by adhesive(s), welding, bonding, overmolding, shrink wrap, etc. Other configurations are also contemplated.

In some embodiments, the implant delivery device 300 may comprise an outer sleeve 370 movably disposed about the elongate shaft 340. For example, the elongate shaft 340 may be movably disposed within a lumen of the outer sleeve 370. In some embodiments, the outer sleeve 370 may be axially slidable about and/or relative to the elongate shaft 340. In some embodiments, the implant delivery device 300 may comprise an implant sheath 372 fixedly attached to a distal end of the outer sleeve 370. In some embodiments, the implant sheath 372 may be configured to constrain the support frame 350 and/or the implant 360 in a delivery configuration, as seen in FIG. 5. In some embodiments, the support frame 350 and/or the implant 360 may be movably disposed within a lumen of the implant sheath 372. The support frame 350 and/or the implant 360 may be configured to expand for deployment at a treatment site when unconstrained by the implant sheath 372 and/or when disposed outside of the lumen of the implant sheath 372, as seen in FIG. 6.

In at least some embodiments, the implant sheath 372 may have an outer diameter that is greater than an outer diameter of the outer sleeve 370. In at least some embodiments, the lumen of the implant sheath 372 may have an inner diameter that is greater than an inner diameter of the lumen of the outer sleeve 370. In some alternative embodiments, the implant sheath 372 may have an outer diameter that is substantially equal to an outer diameter of the outer sleeve 370. In some alternative embodiments, the lumen of the implant sheath 372 may have an inner diameter that is substantially equal to an inner diameter of the lumen of the outer sleeve 370. Other configurations are also contemplated.

In some embodiments, the outer sleeve 370 may be operatively coupled to the actuator 320. In some embodiments, the actuator 320 may be configured to translate and/or slide the outer sleeve 370 axially relative to the elongate shaft 340.

In some embodiments, the medical device system 400 may comprise a bone anchor insertion device 410 having a bone anchor 420 disposed thereon and/or mounted on and/or at a distal end thereof. In some embodiments, the bone anchor insertion device 410 may be similar to the bone anchor insert 150 described above. In some embodiments, the bone anchor insertion device 410 may comprise an elongate shaft 412, a head 414 disposed at a proximal end of the elongate shaft 412, and one or more arms (not explicitly shown) at a distal end of the elongate shaft 412 to retain the bone anchor 420. The lumen 342 of the elongate shaft 340 may be configured to slidably receive the bone anchor insertion device 410 having the bone anchor 420 thereon through the lumen 342 of the elongate shaft 340. In at least some embodiments, the bone anchor 420 and the bone anchor insertion device 410 are advanceable through the lumen 342 of the elongate shaft 340 to secure the implant 360 coupled to the support frame 350 to a bone (e.g., the humerus 16) with the bone anchor 420, as described herein (e.g., FIG. 8).

In some embodiments, the medical device system 400 may further comprise a bone punch similar to the bone punch 140 described above. In some embodiments, the bone punch may comprise an elongate shaft, a head disposed at a proximal end of the elongate shaft, and a piercing tip disposed at a distal end of the elongate shaft. In some embodiments, the bone punch and/or the piercing tip may include two piercing tips. In some embodiments, the bone punch may be configured to form one or more holes in the bone (e.g., the humerus 16) into which the bone anchor 420 may be inserted and/or deployed. In at least some embodiments, the bone punch is advanceable through the lumen 342 of the elongate shaft 340 to the treatment site.

The bone punch may be constructed, except for the piercing tip at the distal end thereof, substantially similar in size and/or shape to the bone anchor insertion device 410 such that both devices may be disposable and/or advanceable within the lumen 342 of the elongate shaft 340. Furthermore, the bone punch and the bone anchor insertion device 410 may engage with and/or function with the retractor 330 in similar ways and/or manners.

After positioning and/or advancing the bone punch within the lumen 342 of the elongate shaft 340, a distal force may be applied to the head of the bone punch with a mallet to urge the piercing tip into bone (e.g., the humerus 16)-similar to the bone anchor insertion device 410 shown in FIG. 7. The head of the bone punch (similar to the head 414 of the bone anchor insertion device 410 shown in FIG. 7) may engage with a proximal ejection block 332 coupled to a linkage 334 engaged with the retractor 330. The linkage 334 may be pivotably engaged with the handle 310 and/or the elongate shaft 340 at a distal end of the linkage 334. As the head of the bone punch pushes distally against the proximal ejection block 332, the proximal ejection block 332 and the linkage 334 are shifted distally into and/or with respect to the handle 310, thereby urging and/or shifting the retractor 330 from the ejection position (e.g., FIGS. 5-6) to the extended position (e.g., FIG. 7). The retractor 330 may be configured to remove the distal end of the bone punch (e.g., the piercing tip) from the bone (e.g., the humerus 16). Thereafter, the retractor 330 may be actuated toward and/or into the handle 310 (e.g., shifted from the extended position to the ejection position) of the implant delivery device 300 to retract the bone punch and remove the distal end of the bone punch from the bone (e.g., the humerus 16).

Similar to the bone punch above, after positioning and/or advancing the bone anchor insertion device 410 within the lumen 342 of the elongate shaft 340, a distal force may be applied to the head 414 of the bone anchor insertion device 410 with a mallet to urge the distal end of the bone anchor insertion device 410 and the bone anchor 420 into the bone (e.g., the humerus 16). In at least some embodiments, the bone anchor insertion device 410 may be positioned and/or advanced within the lumen 342 of the elongate shaft 340 after the bone punch has been removed. In some embodiments, the bone anchor insertion device 410 may be used without the bone punch. The head 414 of the bone anchor insertion device 410 may engage with the proximal ejection block 332 coupled to the linkage 334 engaged with the retractor 330. As the head 414 of the bone anchor insertion device 410 pushes distally against the proximal ejection block 332, the proximal ejection block 332 and the linkage 334 are shifted distally into and/or with respect to the handle 310, thereby urging and/or shifting the retractor 330 from the ejection position (e.g., FIGS. 5-6) to the extended position (e.g., FIG. 7). The retractor 330 may be configured to remove the distal end of the bone anchor insertion device 410 from the bone (e.g., the humerus 16). Thereafter, the retractor 330 may be actuated toward and/or into the handle 310 (e.g., shifted from the extended position to the ejection position) of the implant delivery device 300 to retract the bone anchor insertion device 410 and remove the distal end of the bone anchor insertion device 410 from the bone (e.g., the humerus 16).

In some embodiments, the implant delivery device 300 and/or the elongate shaft 340 of the implant delivery device 340 may be positionable at an oblique angle relative to an upper surface of the implant 360 to deploy the implant 360 coupled to the support frame 350 against the bone (e.g., the humerus 16) and/or the tissue (e.g., the distal tendon 24) adjacent to the bone (e.g., the humerus 16). In some embodiments, the bone anchor insertion device 410 and/or the elongate shaft 412 of the bone anchor insertion device 410 may be positionable at an oblique angle relative to an upper surface of the implant 360 to secure the implant 360 coupled to the support frame 350 to the bone (e.g., the humerus 16) with the bone anchor 420, as seen in FIG. 8.

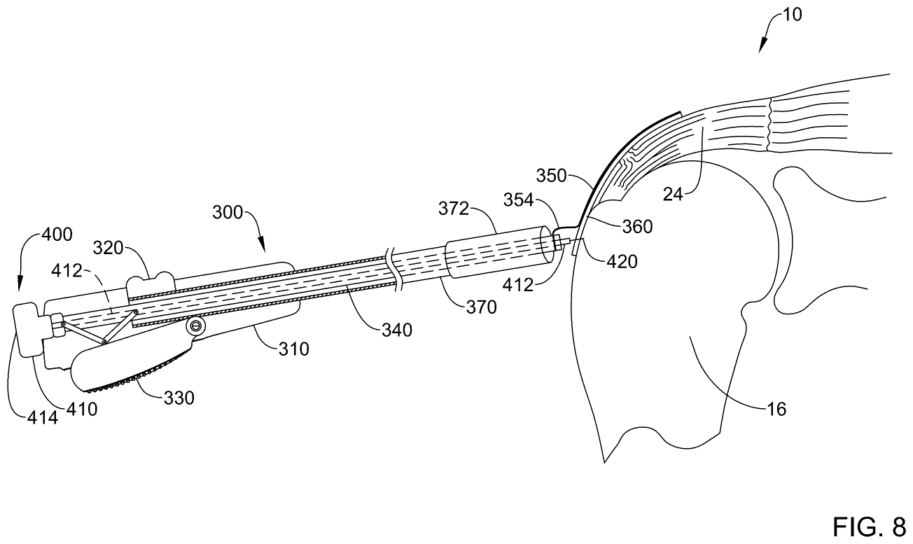

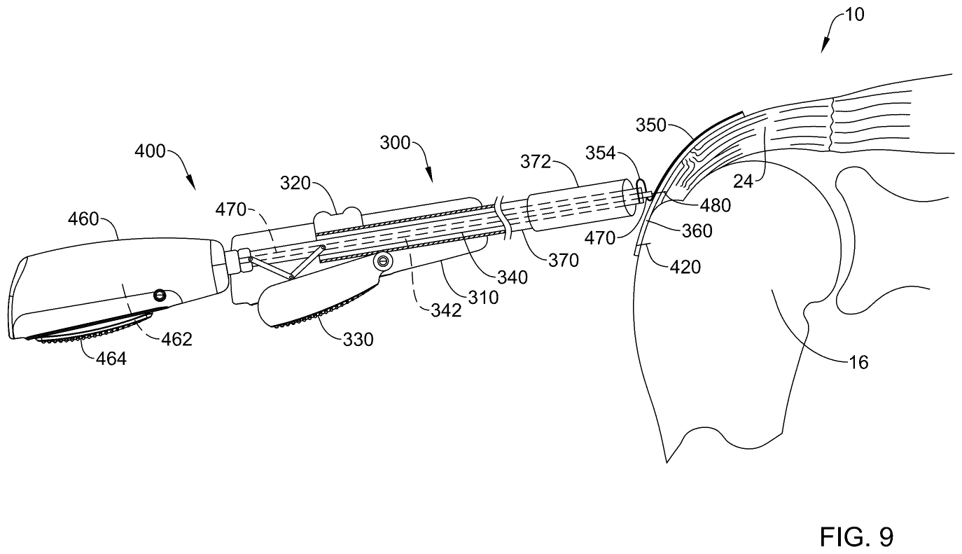

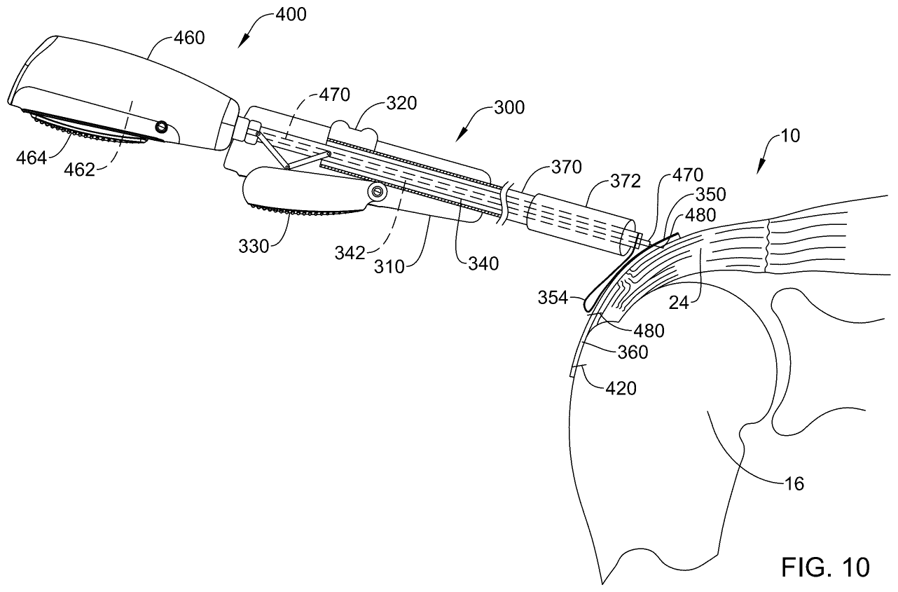

In some embodiments, the medical device system 400 may comprise a tissue anchor delivery device 450 including a handle 460 and an elongate shaft 470 extending distally from the handle 460, as seen in FIGS. 9-10. The tissue anchor delivery device 450 may include at least one tissue anchor 480. In some embodiments, the tissue anchor delivery device 450 may be similar in form and/or function to the tissue anchor delivery device 200 above. The tissue anchor delivery device 450 and/or the elongate shaft 470 may be advanceable through the lumen 342 of the elongate shaft 340 of the implant delivery device 300 to secure the implant 360 coupled to the support frame 350 to tissue (e.g., the distal tendon 24, etc.) adjacent the bone (e.g., the humerus 16) with the at least one tissue anchor 480. The tissue anchor delivery device 450 and/or the elongate shaft 470 may be sized, shaped, and/or configured to extend and/or advance through the lumen 342 of the elongate shaft 340 of the implant delivery device 300. In some embodiments, the at least one tissue anchor 480 may be similar in form and/or function to the tissue anchor(s) 292 above.

In some embodiments, the handle 460 of the tissue anchor delivery device 450 may be disposed at and/or coupled to a proximal end of the elongate shaft 470. In some embodiments, the handle 460 of the tissue anchor delivery device 450 may include an actuation mechanism 462 configured to advance the at least one tissue anchor 480 out of the elongate shaft 470 of the tissue anchor delivery device 450 and/or into the implant 360 and/or tissue at the treatment site and/or within the operative space. The actuation mechanism 462 may be disposed within an interior of the tissue anchor delivery device 450, the handle 460, and/or the elongate shaft 470. In some embodiments, a distal force may be applied to the tissue anchor delivery device 450 and/or the handle 460 of the tissue anchor delivery device 450 to urge the distal end of the tissue anchor delivery device 450 and/or the elongate shaft 470 of the tissue anchor delivery device 450 and the at least one tissue anchor 480 into the implant 360 and/or tissue (e.g., the distal tendon 24, etc.) adjacent the bone (e.g., the humerus 16). In some embodiments, the distal force may be applied with a mallet. Other configurations are also contemplated.

In some embodiments, the elongate shaft 470 of the tissue anchor delivery device 450 may include two tines extending distally away from the distal end thereof. The two tines may extend parallel to the central longitudinal axis of the elongate shaft 470. Collectively, the two tines may define and/or border a passage through which the at least one tissue anchor 480 may pass through as the at least one tissue anchor 480 is advanced and/or deployed out of the elongate shaft 470 of the tissue anchor delivery device 450. The two tines may create pilot holes at the treatment site (e.g., within tissue and/or the implant 360) via application of a distal force to the tissue anchor delivery device 450, wherein the at least one tissue anchor 480 is inserted into the pilot holes upon actuation of the actuation mechanism 462 of the tissue anchor delivery device 450.

In some embodiments, the tissue anchor delivery device 450 and/or the actuation mechanism 462 of the tissue anchor delivery device 450 may include a trigger 464 configured to actuate the actuation mechanism 462. The trigger 464 may be movable relative to the handle 460 of the tissue anchor delivery device 450 to actuate the actuation mechanism 462 to deploy the at least one tissue anchor 480 into the implant 360 and/or tissue (e.g., the distal tendon 24, etc.) adjacent the bone (e.g., the humerus 16). In some embodiments, the trigger 464 may be a lever, and is illustrated in FIGS. 9 and 10 in an actuated position (e.g., after actuation of the actuation mechanism 462), wherein the trigger 464 has been squeezed and/or moved toward and/or into the handle 460 of the tissue anchor delivery device 450. In one example, the trigger 464 may be pivotably coupled to the handle 460 of the tissue anchor delivery device 450. Other configurations are also contemplated.

In some embodiments, the support frame 350, while coupled to the implant 360 and the distal end 344 of the elongate shaft 340, may be configured to permit movement of the distal end 344 of the elongate shaft 340 relative to the implant 360 after the implant 360 is secured to the bone (e.g., the humerus 16) at the treatment site, as seen in FIGS. 9-10. In some embodiments, the support frame 350 is configured to permit the distal end of the elongate shaft 340 to move relative to the implant 360 after the implant 360 is secured to the bone (e.g., the humerus 16) with the bone anchor 420. In some embodiments, the support frame 350, while coupled to the implant 360 and the distal end 344 of the elongate shaft 340, may be configured to permit the distal end 344 of the elongate shaft 340 to move over an entirety of the upper surface of the implant 360 after the implant 360 is secured to the bone (e.g., the humerus 16) at the treatment site.

In some embodiments, the proximal extension 354 of the support frame 350 may be flexible. In some embodiments, the proximal extension 354 of the support frame 350 may be elastic and/or at least partially elastic. In some embodiments, the proximal extension 354 of the support frame 350 may be configured (via selected structure, properties, and/or characteristics) to guide and/or direct the support frame 350 and/or the implant 360 toward and/or to the treatment site (e.g., toward the bone and/or the tissue adjacent the bone) when the implant 360 is not secured to the bone, while also permitting movement of the distal end 344 of the elongate shaft 340 relative to the implant 360 after the implant 360 is secured to the bone (e.g., the humerus 16) at the treatment site.

In some embodiments, the support frame 350 and/or the proximal extension 354 of the support frame 350, while coupled to the implant 360 and the distal end 344 of the elongate shaft 340, may be configured to permit movement of the distal end of the elongate shaft 470 of the tissue anchor delivery device 450 relative to the implant 360 after the implant 360 is secured to the bone (e.g., the humerus 16) at the treatment site while and/or as the distal end 344 of the elongate shaft 340 is movable relative to the implant 360 after the implant 360 is secured to the bone (e.g., the humerus 16) at the treatment site, as seen in FIGS. 9-10. In some embodiments, the support frame 350 and/or the proximal extension 354 of the support frame 350 is configured to permit the distal end of the elongate shaft 470 of the tissue anchor delivery device 450 relative to the implant 360 after the implant 360 is secured to the bone (e.g., the humerus 16) at the treatment site while and/or as the distal end 344 of the elongate shaft 340 is movable relative to the implant 360 after the implant 360 is secured to the bone (e.g., the humerus 16) with the bone anchor 420. In some embodiments, the support frame 350 and/or the proximal extension 354 of the support frame 350, while coupled to the implant 360 and the distal end 344 of the elongate shaft 340, may be configured to permit the distal end of the elongate shaft 470 of the tissue anchor delivery device 450 to move over an entirety of the upper surface of the implant 360 after the implant 360 is secured to the bone (e.g., the humerus 16) at the treatment site while and/or as the distal end 344 of the elongate shaft 340 is movable over the entirety of the upper surface of the implant 360 after the implant 360 is secured to the bone (e.g., the humerus 16) at the treatment site.

In some embodiments, a method of attaching the implant 360 at a treatment site may comprise advancing the implant 360 to the treatment site with the implant delivery device 300, as seen in FIGS. 5 and 8.

In some embodiments, the method of attaching the implant 360 at the treatment site may comprise deploying the implant 360 at the treatment site with the support frame 350 coupled to the distal end 344 of the elongate shaft 340 of the implant delivery device 300, as seen in FIGS. 6 and 8. In some embodiments, the method of attaching the implant 360 at the treatment site may comprise deploying the implant 360 at the treatment site against and/or adjacent bone (e.g., the humerus 16) and/or tissue (e.g., the distal tendon 24) adjacent the bone (e.g., the humerus 16).

In some embodiments, deploying the implant 360 at the treatment site may comprise sliding the outer sleeve 370 of the implant delivery device 300 proximally relative to the elongate shaft 340 of the implant delivery device 300 and the support frame 350 of the implant delivery device 300 using the actuator 320 of the implant delivery device 300. In one example, deploying the implant 360 at the treatment site may comprise sliding the outer sleeve 370 of the implant delivery device 300 proximally relative to the elongate shaft 340 of the implant delivery device 300 and the support frame 350 of the implant delivery device 300 by shifting the actuator 320 of the implant delivery device 300 proximally relative to the handle 310 of the implant delivery device 300. Other configurations, motions, and/or methods are also contemplated in accordance with the configurations and/or embodiments of the actuator 320 disclosed herein.

In some embodiments, the method of attaching the implant 360 at the treatment site may comprise advancing the bone anchor 420 on the distal end of the bone anchor insertion device 410 through the elongate shaft 340 of the implant delivery device 300 to secure the implant 360 to bone (e.g., the humerus 16) with the bone anchor 420 while the support frame 350 remains coupled to the distal end 344 of the elongate shaft 340 of the implant delivery device 300 and the implant 360, as seen in FIG. 8. In some embodiments, the method of attaching the implant 360 at the treatment site may comprise advancing the bone anchor 420 on the distal end of the elongate shaft 412 of the bone anchor insertion device 410 through the elongate shaft 340 of the implant delivery device 300 to secure the implant 360 to bone (e.g., the humerus 16) with the bone anchor 420 while the support frame 350 remains coupled to the distal end 344 of the elongate shaft 340 of the implant delivery device 300 and the implant 360.

In some embodiments, the method of attaching the implant 360 at the treatment site may comprise removing the distal end of the bone anchor insertion device 410 from the bone (e.g., the humerus 16) with the retractor 330 disposed in the handle 310 of the implant delivery device 300. In some embodiments, removing the distal end of the bone anchor insertion device 410 from the bone (e.g., the humerus 16) with the retractor 330 may comprise actuating the retractor 330 from the extended position toward and/or into the handle 310 of the implant delivery device 300 toward and/or to the ejection position to retract the bone anchor insertion device 410 and remove the distal end of the bone anchor insertion device 410 from the bone (e.g., the humerus 16).

In some embodiments, actuating the retractor 330 from the extended position toward and/or into the handle 310 of the implant delivery device 300 toward and/or to the ejection position may be configured to actuate the linkage 334 and the proximal ejection block 332 to apply a proximal force against the head 414 of the bone anchor insertion device 410, thereby pulling and/or retracting the distal end of the bone anchor insertion device 410 from the bone (e.g., the humerus 16).

In some embodiments, the method of attaching the implant 360 at the treatment site may comprise then removing the bone anchor insertion device 410 from the elongate shaft 340 of the implant delivery device 300.

In some embodiments, the method of attaching the implant 360 at the treatment site may comprise reloading the bone anchor insertion device 410 with a second bone anchor disposed on the distal end of the elongate shaft 412 of the bone anchor insertion device 410, and thereafter, advancing the second bone anchor on the distal end of the elongate shaft 412 of the bone anchor insertion device 410 through the elongate shaft 340 of the implant delivery device 300 to secure the implant 360 to bone (e.g., the humerus 16) with the second bone anchor while the support frame 350 remains coupled to the distal end 344 of the elongate shaft 340 and the implant 360. The above discussed process of removing the distal end of the bone anchor insertion device 410 from the bone (e.g., the humerus 16) may be repeated for each time an additional bone anchor is advanced through the elongate shaft 340 of the implant delivery device 300 to secure the implant 360 to bone (e.g., the humerus 16).

In some embodiments, the method of attaching the implant 360 at the treatment site may comprise, after removing the bone anchor insertion device 410 from the elongate shaft 340 of the implant delivery device 300, inserting the tissue anchor delivery device 450 including at least one tissue anchor 480 through the elongate shaft 340 of the implant delivery device 300 to secure the implant 360 to tissue (e.g., the distal tendon 24) adjacent the bone (e.g., the humerus 16) with the at least one tissue anchor 480 while the support frame 350 remains coupled to the distal end 344 of the elongate shaft 340 of the implant delivery device 300 and the implant 360, as seen in FIG. 9.

In some embodiments, the method of attaching the implant 360 at the treatment site may comprise, after deploying a first tissue anchor into tissue (e.g., the distal tendon 24) adjacent the bone (e.g., the humerus 16), moving the distal end of the elongate shaft 340 of the implant delivery device 300 and the distal end of the tissue anchor delivery device 450 relative to the implant 360 while the support frame 350 remains coupled to the implant 360 and the distal end 344 of the elongate shaft 340 of the implant delivery device 300, as seen in FIG. 10, to secure the implant 360 to tissue (e.g., the distal tendon 24) adjacent the bone (e.g., the humerus 16) with the at least one tissue anchor 480 (e.g., a second tissue anchor) at a location spaced apart from the first tissue anchor. In some embodiments, the implant 360 may be secured to tissue (e.g., the distal tendon 24) adjacent the bone (e.g., the humerus 16) with the at least one tissue anchor 480 (e.g., the first tissue anchor, the second tissue anchor, etc.) at an opposite end of the implant 360 from the bone anchor 420, and/or the first bone anchor and the second bone anchor, while the support frame 350 remains coupled to the distal end 344 of the elongate shaft 340 of the implant delivery device 300 and the implant 360.

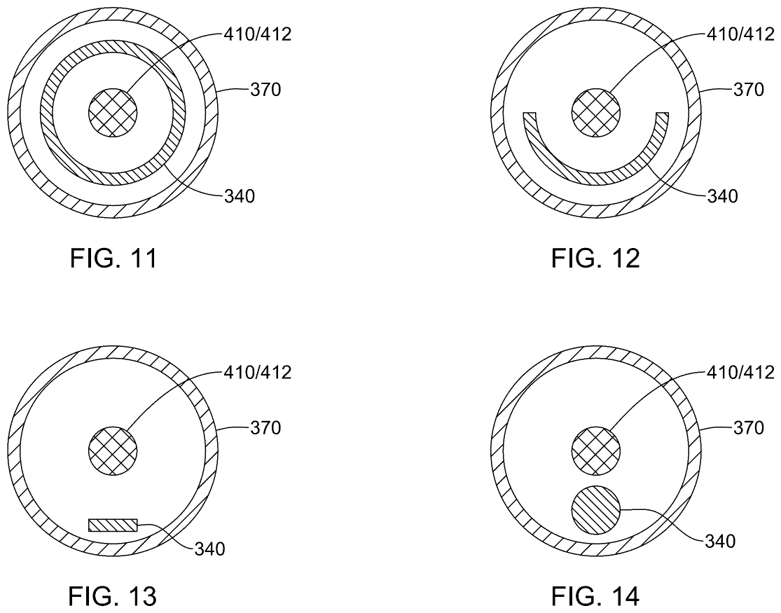

FIGS. 11-14 are cross-sectional views illustrating selected aspects of alternative embodiments and/or configurations for the elongate shaft 340 of the implant delivery device 300. In each figure, the elongate shaft 412 of the bone anchor insertion device 410 is shown in schematic cross-section for reference. In FIG. 11, the elongate shaft 340 of the implant delivery device 300 is shown having a hollow circular cross-section, such as when formed as and/or from a tubular member. In the embodiment of FIG. 11, the elongate shaft 412 of the bone anchor insertion device 410 may be disposed within the elongate shaft 340 of the implant delivery device 300.

In FIG. 12, the elongate shaft 340 of the implant delivery device 300 is shown having a semi-circular cross-section and/or a concave cross-section, such as when formed as and/or from a partial tubular member. In the embodiment of FIG. 12, the elongate shaft 412 of the bone anchor insertion device 410 may be disposed alongside the elongate shaft 340 of the implant delivery device 300 within the outer sleeve 370 and/or a concave side of the elongate shaft 340 of the implant delivery device 300 may face towards the elongate shaft 412 of the bone anchor insertion device 410 within the outer sleeve 370.

In FIG. 13, the elongate shaft 340 of the implant delivery device 300 is shown having a rectangular cross-section. In some embodiments, the elongate shaft 340 of the implant delivery device 300 may be formed with a different polygonal cross-sectional shape, such as square, triangular, hexagonal, octagonal, etc. In the embodiment of FIG. 13, the elongate shaft 412 of the bone anchor insertion device 410 may be disposed alongside the elongate shaft 340 of the implant delivery device 300 within the outer sleeve 370.

In FIG. 14, the elongate shaft 340 of the implant delivery device 300 is shown having a solid circular cross-section, such as when formed as and/or from a shaft or rod. In some embodiments, the elongate shaft 340 of the implant delivery device 300 may be formed with a different rounded cross-sectional shape, such as oval, egg-shaped, etc. In the embodiment of FIG. 14, the elongate shaft 412 of the bone anchor insertion device 410 may be disposed alongside the elongate shaft 340 of the implant delivery device 300 within the outer sleeve 370.

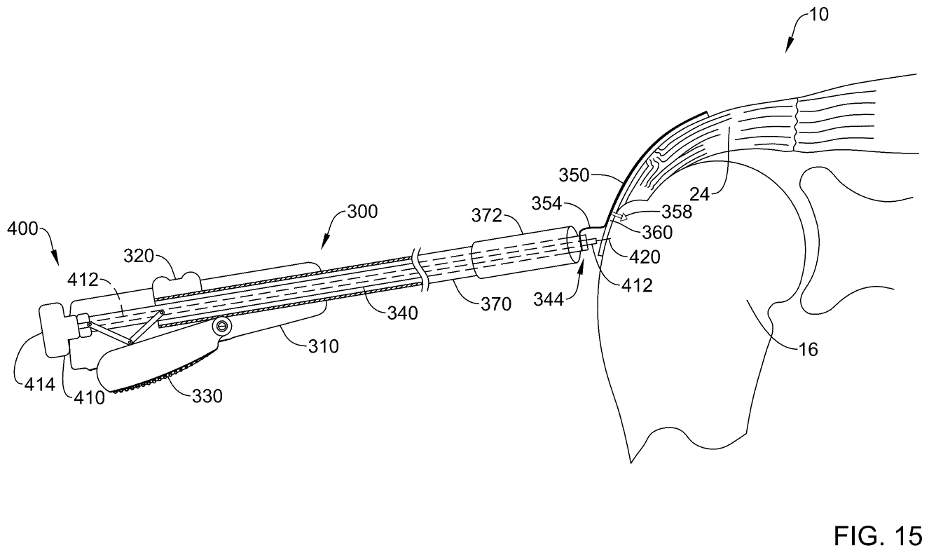

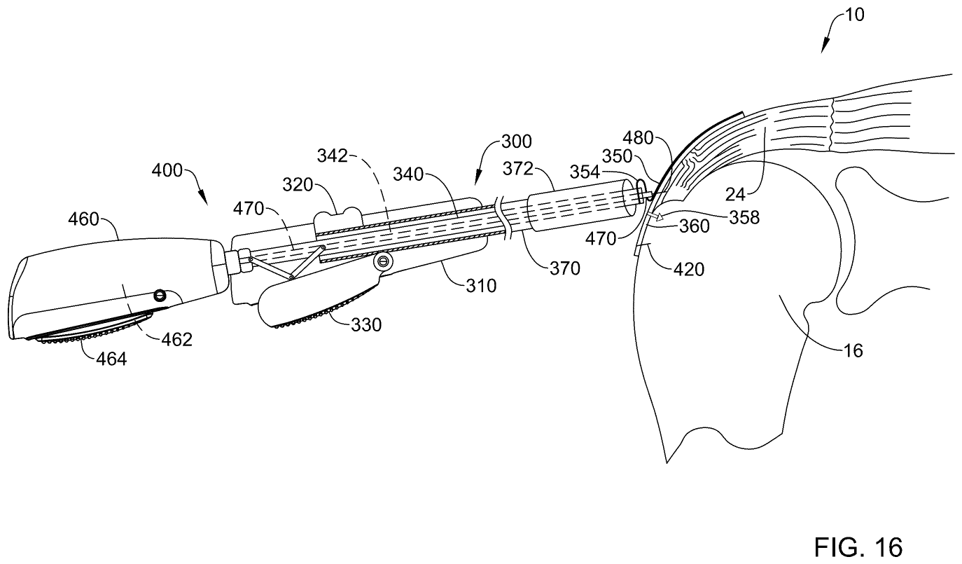

FIGS. 15-16 illustrate an alternative configuration of FIGS. 8-9 above. In some embodiments, the support frame 350 may comprise a tack member 358 extending therefrom and/or extending through the implant 360. In some embodiments, the tack member 358 may be configured to provide temporary fixation of the support frame 350 and the implant 360 to bone (e.g., the humerus 16) and/or tissue (e.g., the distal tendon 24) at the treatment site. As shown, the tack member 358 may be configured to hold the support frame 350 and the implant 360 in place as the bone anchor 420 and/or the at least one tissue anchor 480 is deployed. In some embodiments, the support frame 350 and/or the proximal extension 354 of the support frame 350 may be configured to permit movement of the distal end 344 of the elongate shaft 340 relative to the implant 360 after the implant 360 is secured to the bone (e.g., the humerus 16) at the treatment site, as discussed herein. After attaching the implant 360 to bone (e.g., the humerus 16) and/or tissue (e.g., the distal tendon 24) at the treatment site, the support frame 350 may be removed along with the implant delivery device 300. When removing the support frame 350, the tack member 358 may also be removed from the implant 360 and/or the treatment site. Since the support frame 350 is fixedly secured and/or fixedly attached to the distal end 344 of the elongate shaft 340 of the implant delivery device 300, no tether is used or necessary to remove the support frame 350 and the tack member 358 after attaching the implant 360 to bone (e.g., the humerus 16) and/or tissue (e.g., the distal tendon 24) at the treatment site.

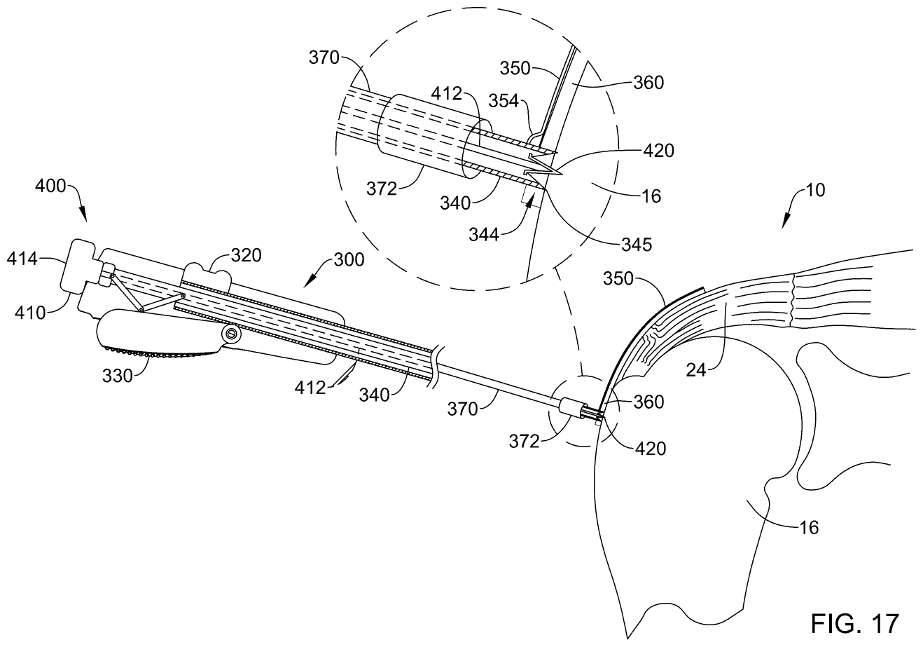

FIG. 17 schematically illustrates selected aspects of an alternative configuration of the implant delivery device 300. In at least some embodiments, the implant delivery device 300 may be constructed and/or may function as described above, except as explicitly described below. Accordingly, each and every feature and/or detail is not repeated herein.

In some embodiments, the distal end 344 of the elongate shaft 340 of the implant delivery device 300 may be configured to pierce the implant 360 and engage the bone (e.g., the humerus 16) to temporarily fix the implant 360 to the bone (e.g., the humerus 16). In some embodiments, the distal end 344 of the elongate shaft 340 of the implant delivery device 300 may be configured to pierce the implant 360 and engage the bone (e.g., the humerus 16) to temporarily fix the implant 360 to the bone (e.g., the humerus 16) as the bone anchor 420 is deployed and/or secures the implant 360 to the bone (e.g., the humerus 16) while the support frame 350 of the implant delivery device 300 remains coupled to the elongate shaft 340 of the implant delivery device 300 and the implant 360. In some embodiments, the distal end 344 of the elongate shaft 340 of the implant delivery device 300 may comprise at least one piercing element 345 disposed at a distalmost end of the elongate shaft 340 of the implant delivery device 300. In some embodiments, the at least one piercing element 345 may be formed by modifying the distal end 344 of the elongate shaft 340 of the implant delivery device 300, such as be cutting, molding, removing material, etc. In some embodiments, the at least one piercing element 345 may comprise a plurality of chisel points formed at the distalmost end of the elongate shaft 340 of the implant delivery device 300. In some embodiments, the at least one piercing element 345 and/or the plurality of chisel points may be spaced apart around the circumference of the elongate shaft 340 of the implant delivery device 300. Other configurations are also contemplated.

The materials that can be used for the various components of the medical devices, systems, and various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the implant delivery device, the bone anchor(s), the bone anchor insertion device, the tissue anchor(s), the tissue anchor delivery device, etc., and/or elements or components thereof.

In some embodiments, the system and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA, for example PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, portions or all of the system and/or components thereof may be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids a user in determining the location of the system. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-protein and/or anti-bacterial agents (such as 2-methacryroyloxyethyl phosphorylcholine (MPC) and its polymers or copolymers); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An implant delivery device, comprising:
a handle;
an elongate shaft extending distally from the handle; and
a support frame coupled to a distal end of the elongate shaft;
wherein the elongate shaft includes a lumen extending along an entire length of the elongate shaft;
wherein the lumen opens to a proximal end of the handle;
wherein the lumen of the elongate shaft is configured to receive a bone anchor insertion device having a bone anchor thereon through the lumen of the elongate shaft.

2. The implant delivery device of claim 1, further comprising an outer sleeve that is disposed about the elongate shaft and is axially slidable relative to the elongate shaft.

3. The implant delivery device of claim 2, further comprising an implant sheath fixedly attached to a distal end of the outer sleeve.

4. The implant delivery device of claim 3, wherein the support frame is releasably coupled to an implant and the implant sheath is configured to constrain the support frame and the implant in a delivery configuration.

5. The implant delivery device of claim 4, wherein the support frame and the implant are configured to expand for deployment at a treatment site when unconstrained by the implant sheath.

6. The implant delivery device of claim 4, wherein the support frame, while coupled to the implant, is configured to permit movement of the distal end of the elongate shaft relative to the implant after the implant is secured to bone at the treatment site.

7. The implant delivery device of claim 8, wherein the support frame, while coupled to the implant, is configured to permit the distal end of the elongate shaft to move over an entirety of an upper surface of the implant after the implant is secured to bone at the treatment site.

8. The implant delivery device of claim 1, wherein the handle includes an actuator configured to slide the outer sleeve axially relative to the elongate shaft.

9. The implant delivery device of claim 1, wherein the handle includes a retractor configured to remove a distal end of the bone anchor insertion device from bone.

10. A medical device system, comprising:
an implant delivery device, comprising:
a handle;
an elongate shaft extending distally from the handle; and
a support frame coupled to a distal end of the elongate shaft;
wherein the elongate shaft includes a lumen extending along an entire length of the elongate shaft;
wherein the lumen opens to a proximal end of the handle; and
a bone anchor insertion device having a bone anchor mounted on a distal end thereof;
wherein the bone anchor and the bone anchor insertion device are advanceable through the lumen of the elongate shaft to secure an implant coupled to the support frame to a bone with the bone anchor.

11. The medical device system of claim 10, further comprising a tissue anchor delivery device including at least one tissue anchor, wherein the tissue anchor delivery device is advanceable through the lumen of the elongate shaft to secure the implant coupled to the support frame to tissue adjacent the bone with the at least one tissue anchor.

12. The medical device system of claim 10, wherein the support frame is configured to permit the distal end of the elongate shaft to move relative to the implant after the implant is secured to the bone with the bone anchor.

13. The medical device system of claim 10, wherein the support frame extends distally from the distal end of the elongate shaft.

14. The medical device system of claim 10, wherein the bone anchor insertion device is positionable at an oblique angle relative to an upper surface of the implant to secure the implant coupled to the support frame to the bone with the bone anchor.

15. A method of attaching an implant at a treatment site, comprising:
advancing the implant to the treatment site with an implant delivery device;
deploying the implant at the treatment site with a support frame coupled to a distal end of an elongate shaft of the implant delivery device; and
advancing a bone anchor on a distal end of a bone anchor insertion device through the elongate shaft of the implant delivery device to secure the implant to bone with the bone anchor while the support frame remains coupled to the elongate shaft and the implant.

16. The method of claim 15, wherein deploying the implant at the treatment site comprises sliding an outer sleeve of the implant delivery device proximally relative to the elongate shaft and the support frame using an actuator of the implant delivery device.

17. The method of claim 15, further comprising:
removing the distal end of the bone anchor insertion device from bone with a retractor disposed in a handle of the implant delivery device, and then removing the bone anchor insertion device from the elongate shaft of the implant delivery device.

18. The method of claim 17, further comprising:
after removing the bone anchor insertion device from the elongate shaft of the implant delivery device, inserting a tissue anchor delivery device including at least one tissue anchor through the elongate shaft to secure the implant to tissue adjacent the bone with the at least one tissue anchor while the support frame remains coupled to the elongate shaft and the implant.

19. The method of claim 18, wherein the implant is secured to tissue adjacent the bone with the at least one tissue anchor at an opposite end of the implant from the bone anchor while the support frame remains coupled to the elongate shaft and the implant.

20. The method of claim 15, wherein the distal end of the elongate shaft is configured to pierce the implant and engage the bone to temporarily fix the implant to the bone as the bone anchor secures the implant to the bone while the support frame remains coupled to the elongate shaft and the implant.
